(19) United States
(12) Patent Application Publication
(10) Pub. No.: US 2004/0186082 A1
(43) Pub. Date: Sep. 23, 2004

(54) Enhanced phototherapy for the treatment of cancer and autoimmune disease

(76) Inventor: Raymond A. Hartman, Carlsbad, CA (US)

Correspondence Address: GORDON & REES LLP, 101 WEST BROADWAY, SUITE 1600, SAN DIEGO, CA, 92101, US

(21) Appl. No.: 10/393,716

(22) Filed: Mar. 20, 2003

(51) Int. Cl. A61K 31/59; A61K 31/355; A61K 31/557

(52) U.S. Cl. 514/167; 514/458; 514/562; 514/573

(57) ABSTRACT

The invention is a method for the treatment of autoimmune disease and cancer comprising the application of vitamin D precursors followed by UVA or UVB or exposure to blue light. The method can also include sequential or concurrent treatment with UVB irradiation and UVA irradiation or exposure to blue light. The invention is the vitamin D precursor compositions used in the invention.

### BACKGROUND OF THE INVENTION

Phototherapy is the use of UVA (320-390 nm) and UVB (290-320 nm) light for the treatment of dermatological conditions such as psoriasis, eczema, vitiligo and other autoimmune (generally T-cell mediated) conditions. The history of phototherapy goes back several thousand years to the use of solar radiation in combination with application of natural products (e.g. plant extracts, coal tar) to the skin to relieve dermatological conditions. A watershed development in 1925 was the introduction of the Goeckerman treatment for psoriasis which involves repeatedly applying coal tar to a patient's body and exposing the patient to solar radiation. This treatment is still in use today.

Solar light sources were eventually replaced with lamp sources, and booths were developed for total body radiation as well as lamp boxes for limb radiation. Light sources were optimized to deliver radiation at more optimal wavelengths to improve the immunosuppressive effects of radiation. Therapies based on UVA irradiation became useful when exposure to UVA was combined with pre-treatment with topical photosensitizers, namely the plant based compound psoralen, making it an effective therapy. The treatment became known as psoralen UVA (PUVA) or photochemotherapy. A putative mechanism of action for PUVA is that the psoralen intercalates into the DNA and irradiation generates photoadducts resulting in DNA damage, decreasing protein synthesis and cell growth. It has been alternatively proposed that PUVA may alter the integrity of cell membranes or result in the cross-linking of proteins. To date, no consensus mechanism has been defined.

The dose of radiation used in phototherapy is limited by the exposure of healthy skin to UV radiation. Healthy tissue generally burns more easily than diseased tissue and thus limits the amount of radiation that can be delivered in a single session. In UVB therapy, the usual limiting dose is called 1 minimum erythemal dose (MED) and in PUVA, the usual limiting dose is called 1 minimum phototoxic dose (MPD). Due to the radiation dose limitations of healthy tissues, both UVB and PUVA therapy require a large number of treatments. Localized delivery of UV using a focused phototherapy apparatus (e.g. see Hartman, U.S. Pat. No. 6,413,268) allows for a reduction in the number of treatments by limiting delivery of UV to normal tissue. However, regardless of the UV delivery method, UV treatment modalities have variable levels of success in any given cohort of patients. This is partially due to the fact that the effects of the radiation on the diseased tissue are not fully understood. Treatment modalities have been developed empirically rather than rationally.

The limitations of current UVB phototherapy are exemplified in the UVB treatment of vitiligo. Due to the non-pigmented nature of the skin, only very low UVB doses can be used initially with a gradual increase in follow-on procedures to prevent burning. No topical augmentation is used. Typically hundreds of treatments are required to achieve marginal repigmentation.

In addition to phototherapy, pharmacological interventions have also been developed for the treatment of autoimmune skin disorders such as vitiligo and psoriasis. These disorders are characterized by abnormal T-cell activity, cytokine (interleukin and interferon) imbalances and autocrine and paracrine imbalances. Treatment methodologies that regulate cytokine production offer some relief to these disease conditions. Calcitriol (1,25 dihydroxyvitamin D3), a regulator of a number of cytokines and inflammatory mediators, is one such agent.

Calcitriol is also known to be a regulator of apoptosis and has been used as a chemotherapuetic agent in the treatment of cancer. Exogenous calcitriol has been shown to be effective in restoring some of the apoptotic programming to cancer cells and increasing clearance of cancer cells by the immune system. However, the amounts of calcitriol or its non-calcemic analogs required to provide therapeutic treatment can present other medical difficulties.

Oral and topical formulations containing calcitriol have been developed. However, the efficacy and utility of calcitriol is limited by the systemic hypercalcemia caused by the high doses of the drug that are required. Analogs of calcitriol, such as calcipotriol, have been developed to mimic the effects of calcitriol with lower calcemic action. These non-calcemic analogs can be applied in concentrations 20 times higher than calcitriol without leading to hypercalcemia. These topical treatments are considered less effective than phototherapy, but provide some convenience in that they are not time consuming and patients can administer the treatment to themselves at home.

The efficacy of topical calcitriol is limited by its degradation, slow diffusion across the epidermis and a relatively high rate of clearance from the base of the epidermis into the circulatory system. Calcitriol is a large lipophilic molecule that diffuses slowly through the extracellular matrix. When it enters a cell, it activates the production of a calcitriol-inactivating enzyme, 24-hydroxylase within the cell. This enzyme is produced by a cytochrome called CYP24, which is triggered by the presence of calcitriol within the cell. When the calcitriol begins to reach the lower levels of the epidermis, it is removed from the extracellular matrix by vitamin D binding protein (VDBP) into the circulation. During its transit time through the epidermis the calcitriol is vulnerable to photolysis from any UVB or UVA energy impinging upon the skin. These limitations make it difficult to obtain a substantial concentration of calcitriol throughout the epidermis. Moreover, transcriptional activation by the calcitriol-VDR-RXR heterotrimer is strongly inhibited by both TNF-alpha and the NFkappaB nuclear transcription factor. These factors are usually present in autoimmune and cancerous conditions and are often considered markers of the disorders. Considering all of these factors, it is not surprising that topical calcitriol has only marginal benefit even when used in high concentrations in its non-calcemic forms.

Calcitriol has pleiotropic effects. First, calcitriol alters calcium homeostasis in the cell, affecting a number of signaling pathways. Second, calcitriol effects cell growth, differentiation and cytokine regulation at a genetic level as a component of a trimeric transcription factor further comprised of the vitamin D receptor (VDR) and the retinoid X receptor (RXR). The VDR-RXR-calcitriol heterotrimer binds to nuclear factor of activated T-cell (NFAT) sites and vitamin D response element (VDRE) sites in the DNA, thereby regulating transcription of interleukins and other inflammatory mediators. VDR is generally free in the cytosol and available in cells to form the complex. Calcitriol is also typically unbound when present in the cell. However, RXR is typically bound to retinoids present in the cell; and therefore, commonly the limiting factor in the formation of the heterotrimer. Application of calcitriol does not alter the amount of free RXR present in the cell.

It is known in phototherapy that retinoids are depleted by UV radiation. UV radiation isomerizes or degrades cellular retinoids, and as a result, the retinoic acid binding proteins such as RXR, RAR-RXR and the cellular retinoic acid binding protein (CRABP) become available for binding new ligands. Most retinoid photolysis occurs in the UVB and UVA regions of the spectrum. However, the depletion of all-trans retinoic acid (ATRA) occurs in the blue spectrum with a peak degradation at 420 nm. ATRA is the precursor for 9-cis retinoic acid, the major ligand for RXR.

UV light as also able to stimulate the production of prostaglandin E2 (PGE2). PGE2 levels are elevated in a number of cancers and autoimmune diseases. As with most prostaglandins, PGE2 has pleiotropic effects, one of which is the increased production of VDR.

The concurrent use of UVB or PUVA phototherapy and topical calcitriol or its analogs for treatment of autoimmune disorders has had little success. The combination of the therapies is neither synergistic nor cost-effective. Moreover, the two therapies cannot be combined into a single treatment since calcitriol is photolabile to UVB and UVA radiation. Production of calcitriol in skin from physiological doses (>½ MED) of UVB is very low, measured in femptomolar (10⁻¹⁵ molar) amounts. It is known that topical calcitriol must be applied in nanomolar amounts (10⁻⁹ molar) to have even a moderate physiological effect.

Vitamin D is a precursor to calcitriol. Vitamin D is produced from provitamin D (7-dehydrocholesterol) in the skin upon exposure to UVB (290-315 nm) irradiation. It is known that wavelengths longer than 315 nm (i.e. UVA radiation) are not energetic enough to produce vitamin D from provitamin D. Lehman demonstrated that femptomolar calcitriol is produced in the skin with UVB irradiation from 290-315 nm, and further demonstrated that the origin of the calcitriol was vitamin D produced from provitamin D in the skin. However, due to the low calcitriol production by the UVB, the therapeutic effects of the calcitriol in UVB therapy have been considered negligible. UVB therapy is believed to work mainly by depletion of Langerhans cells (antigen presenting cells) in the irradiated area and/or by induction of suppressor T-cells to mediate the autoimmune reaction. It has also been suggested that UVB acts by suppressing DNA synthesis or by multimerization of cell membrane proteins. No link has been proposed for a common mechanism of action between PUVA and UVB therapy.

A variety of vitamin D precursors and vitamin D analog precursors have been taught for a variety of uses from the treatment of calcium deficiencies to use as a sunblock (e.g. Hollick, U.S. Pat. Nos. 4,230,701; 4,310,511; 5,167,953 and 5,395,829; Dikstein, U.S. Pat. No. 4,610,978; and Hansen, U.S. Pat. No. 4,551,214). None of the patents teach the combination of administration of the compounds with exposure to light for therapeutic purposes.

Methods combining the application of previtamin D or previtamin D analogs with exposure to specific wavelengths of light have been proposed for the prevention and treatment of vitamin D deficiencies. Pre-vitamin D is applied topically (Hollick, U.S. Pat. No. 5,194,248) or administered orally or parenterally (Hollick, U.S. Pat. No. 5,422,099) followed by exposure to light at a wavelength longer than is required to promote the isomerization of previtamin D to vitamin D. The patents teach that previtamin D can be converted to vitamin D with 300 nm light or broad-band 350 or 355 nm light at 0° C. (i.e. non-physiological conditions). The method is not taught for the treatment of skin disease.

### SUMMARY OF THE INVENTION

The invention is a method of treating autoimmune diseases and cancer comprising direct application of compositions comprised of calcitriol and calcitriol analog precursors, optionally containing NFkappaB inhibitors, VDR stimulants and/or photosensitizers, followed by treatment with UV and/or blue light phototherapy. The method comprises irradiation methods using single or multiple bands of radiation either simultaneously or sequentially, using the compositions of the invention. The components of the composition are selected based on the wavelengths of light to be used and on the individual to be treated.

The invention is a method comprising application of trans-previtamin D analogs to the tissue to be treated followed by treatment with UVA or blue light with psoralen and/or other photosensitizing agents for the treatment of disease.

The method of the invention is applicable to the treatment of a number of autoimmune diseases, particularly those of the skin including, but not limited to psoriasis, vitiligo and alopecia. Additionally, the method of the invention is applicable to the treatment of a number of cancers including, but not limited to T-cell lymphoma, melanoma, breast cancer, prostate cancer or any other tumor having a sufficiently solid structure to be treated using the method of the invention. The method comprises the use of any natural or non-natural provitamin D or provitamin D analogs (i.e. calcitriol precursor or calcitriol analog precursor) including, but not limited to 7-dehydrocholesterol and ergosterol. The method comprises the use of ultraviolet light, either UVA or UVB, or blue light which may be administered to the tissue to be treated by any of a number of methods and apparatuses including, but not limited to an apparatus for localized delivery of radiation (e.g. Hartman, U.S. Pat. No. 6,447,537), radiation booths, limb boxes or other delivery devices. Irradiation or blue light may be delivered directly through the skin when the cancer is subcutaneous. Alternatively, the irradiation or light can be delivered using a fiberoptic delivery device inserted arthroscopicly into the individual or a more standard light source with open surgical methods. This method is particularly useful in clearing the margins around a surgically removed tumor. The treatment method is most limited by the ability to deliver light or irradiation to the tumor.

The invention is the compositions for use in the method of the invention. Such compositions contain any of a number of ingredients including provitamin D, provitamin D analogs, previtamin D and previtamin D analogs in an ointment, cream or other formulation that can be applied to the tissue to be treated and allow for absorbtion of the active agents by the tissue. Additionally, the compound may contain phototsensitizers and/or TNF-alpha or NfkappaB inhibitors.

### BRIEF DESCRIPTION OF THE DRAWINGS

The present invention will be better understood from the following detailed description of an exemplary embodiment of the invention, taken in conjunction with the accompanying drawings.

FIGS. 1A and 1B show a series of natural and non-natural vitamin D analogs;

FIGS. 2A and 2B show pathways of vitamin D biosynthesis, activation and inactivation; and

FIG. 3 shows the conversion of trans-previtamin D to cis-previtamin D in the presence of a photosensitizing agent and UVA irradiation.

### DETAILED DESCRIPTION AND PREFERRED EMBODIMENTS

### Definitions

UVA radiation is light with a wavelength from 320-390 nm.

UVB radiation is light with a wavelength from 290-320 nm.

Blue light is light with a wavelength from 400-440 nm.

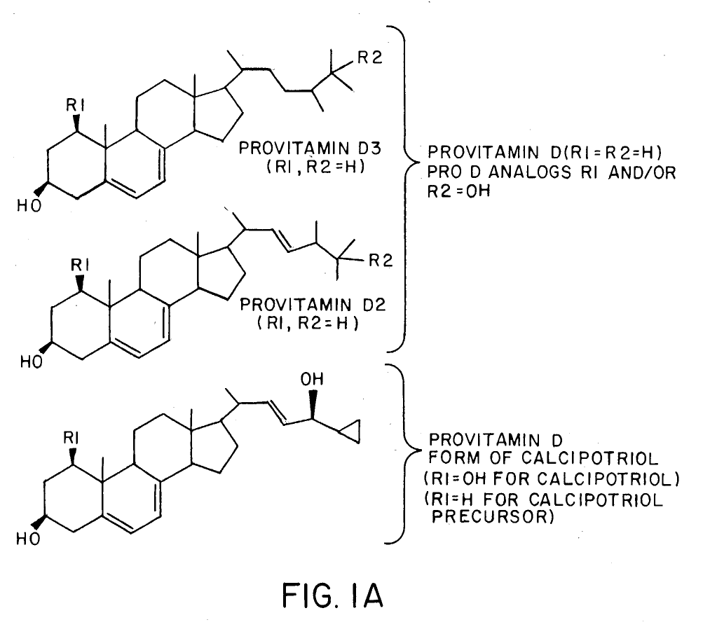

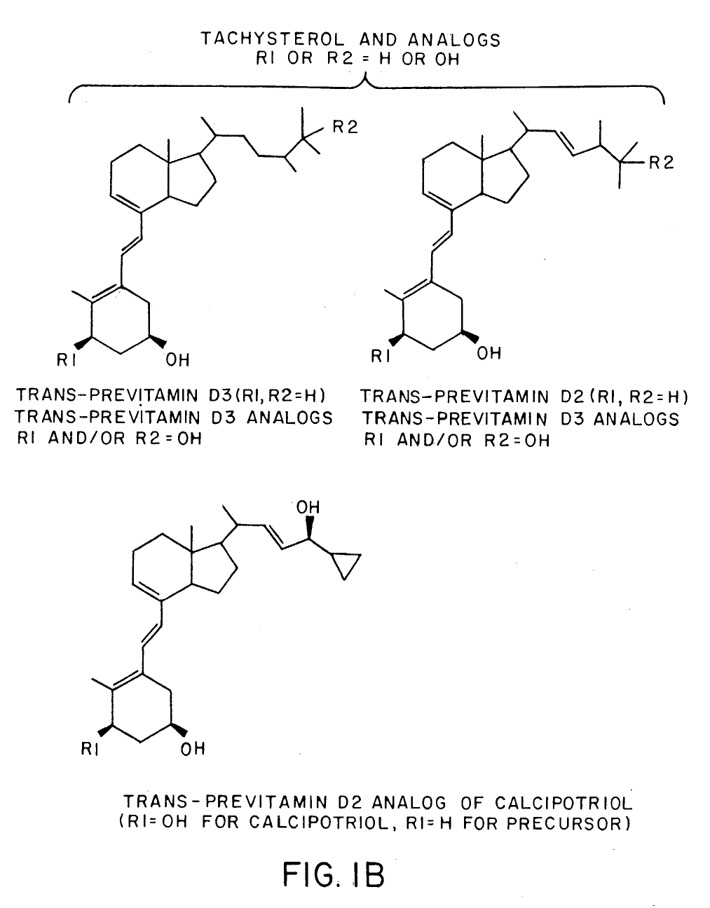

Previtamin D and previtamin D analogs are the natural form of trans-previtamin D₂ and D₃ (tachysterol) or modified forms of trans-previtamin D that upon cis-isomerization with light and a photosensitizer thermally isomerizes directly to calcitriol or the desired calcitriol precursor form or calcitriol analog. Trans-previtamin D analogs include those shown in FIG. 1B.

Provitamin D and provitamin D analogs are compounds that when irradiated with UVB from 290-315 nm and hydroxylated by P450 enzymes (e.g. CYP 25, 27A, 27B) become calcitriol, or calcemic or non-calcemic analogs of calcitriol (e.g. calcipotriol, calcipotriene). In the context of the invention, the term provitamin D should be understood to encompass provitamin D2 (ergosterol) and provitamin D3 (7-dehydrocholesterol) as well as any analogs that would be included in the definition above. There is a simple ring opening reaction caused by UVB light and the thermal isomerization leads directly to the formation of calcitriol or a calcitriol analog. The chemistry and biochemistry of such compounds is well understood as are the actions of the P450 cytochromes. Examples of provitamin D analogs include, but are not limited to those seen in FIG. 1A.

Calcipotriene analog of provitamin D2 is shown in FIG. 1A having a cyclopropyl group at the terminus.

Photosensitizing agents are compounds that enhance the reaction of trans-previtamin D to cis-previtamin D in the presence of UVA or blue light that are safe for application to the body. Photosensitizing agents include, but are not limited to psoralen and psoralen analogs, anthracene or other polycyclic aromatic hydrocarbons (PAH) molecules or compounds, furocoumarins, and thiophene based pharmaceuticals.

Tumor Necrosis Factor (TNF)-alpha inhibitors and Nuclear Factor (NF)kappaB inhibitors include compounds that inhibit the binding of TNF-alpha and NFkappaB to the DNA. This includes both compounds for topical application such as vitamin E succintate, vitamin C, n-acetylcysteine and prostaglandin E2 as well as oral pharmaceuticals such as etanercept and infliximab.

UV therapy and light therapy delivery devices include, but are not limited to focused phototherapy devices such as that taught by Hartman (U.S. Pat. No. 6,413,268), booths, limb irradiation boxes and fiber optic light sources. As shielding is not required with blue light, it may be delivered using any light source that is capable of delivering a specific range of visible light.

### DETAILED DESCRIPTION

The invention is a method of treatment of autoimmune diseases and cancer comprising application of calcitriol precursors to the area to be treated, providing sufficient time for precursor molecules to traverse the tissue, such as the epidermis, and irradiation of the area to be treated with UV and/or blue light. The topical compositions also include compounds to block TNF-alpha and NFkappaB inhibition of binding of the VDR-RXR-calcitriol heterotrimer to DNA.

The invention consists of formulations of different provitamin D and provitamin D analogs, all of which are precursors of calcitriol or calcitriol analogs, to be used with UVB radiation. In another embodiment of the invention, formulations consist of different trans-previtamin D molecules to be used with UVA and blue light radiation along with photosensitizers. In another less preferred embodiment, formulations contain calcitriol or calcitriol analogs for use with blue light radiation for phototherapy. The chemistry of such compounds and the effects of various wavelengths of UV radiation on them are well known to those skilled in the art. Compounds and wavelengths for the treatment of various disorders are selected based on wavelengths known to be most beneficial in the treatment of certain conditions or by other means known to those skilled in the art.

Precursor molecules that can be used are ergosterol which is the precursor to Vitamin D2, and any other provitamin molecules that when irradiated and hydroxylated by P450 enzymes (e.g. CYP 25,27A, 27B) become non-calcemic analogs of calcitriol (see FIG. 1A). It is obvious to those skilled in the art that minor modifications to the precursor molecule to make it less calcemic will not affect the ring opening caused by UVB radiation or the thermal isomerization of the molecule. For example, minor modifications to the provitamin D or ergosterol can easily be made such that the end product is calcipotriol or calcipotriene rather than calcitriol. Such changes are covered in the scope of this patent. Stable intermediates of vitamin D biosynthesis may be used if they are sufficiently stable and have favorable transport characteristics such that they diffuse efficiently throughout the tissue to be treated.

In a preferred embodiment, 7-dehydrocholesterol (provitamin D3) is used due to its very low toxicity, lack of degradation by P450 oxidative enzymes (e.g. CYP24), absence of clearance from the targeted lesional site by specialized proteins, such as VDBP, and thermal stability. This use of 7-dehydrocholesterol avoids the drawbacks of the current use of calcitriol or its derivatives as a topical therapy. Since the compound has no transport or degradation problems like calcitriol the distribution in the tissue of the 7-dehydrocholesterol is relatively homogeneously throughout the tissue. Vitamin E succinate and/or vitamin C are administered with the 7-dehydrocholesterol to increase the efficacy of the therapy.

The method of the invention can include treatment at a second wavelength of light, either UVA or blue light. The method may additionally include treatment with a photosensitizing agent prior to treatment with the second wavelength of light. The choice to include the additional wavelengths of irradiation in the method of the invention is based on a number of factors including, but not limited to the disease to be treated, the individual to be treated and the availability of materials required to carry out the invention. Similarly, photosensitizing agents and UVA or blue light are chosen based on considerations such as interactions with the specific vitamin D analog or the specific condition being treated. For example, treatment with blue light may be more beneficial to individuals with vitiligo who have no skin pigment and are susceptible to burns with even low energy radiation.

A new potential paradigm for phototherapy and photochemotherapy is presented herein. This paradigm is that both UVB phototherapy and PUVA photochemotherapy achieve the majority of their immunomodulatory therapeutic value from the production and upregulation of VDR-RXR-calcitriol complexes, and their subsequent regulation of transcriptional elements to modulate cytokine and autocrine/paracrine factor production.

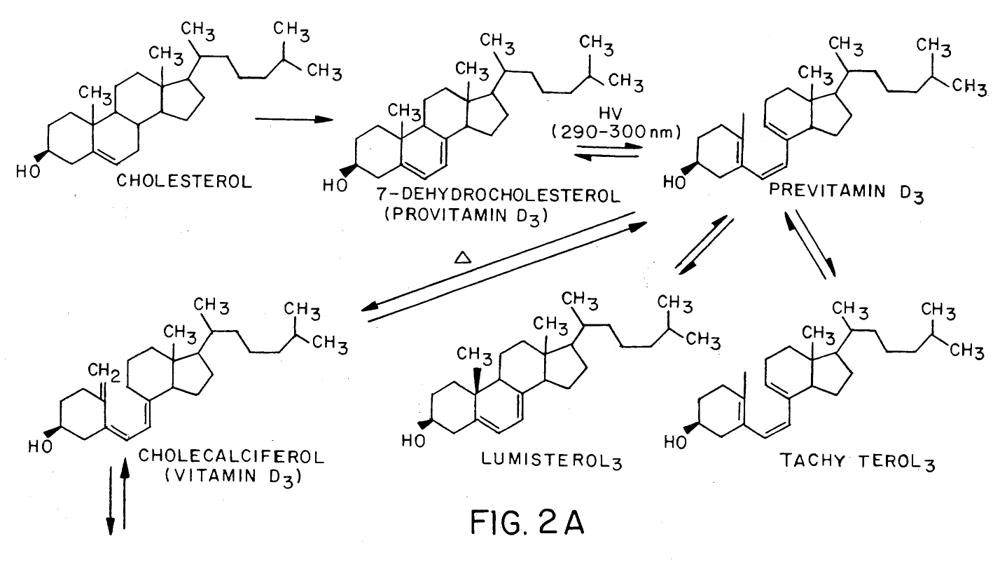

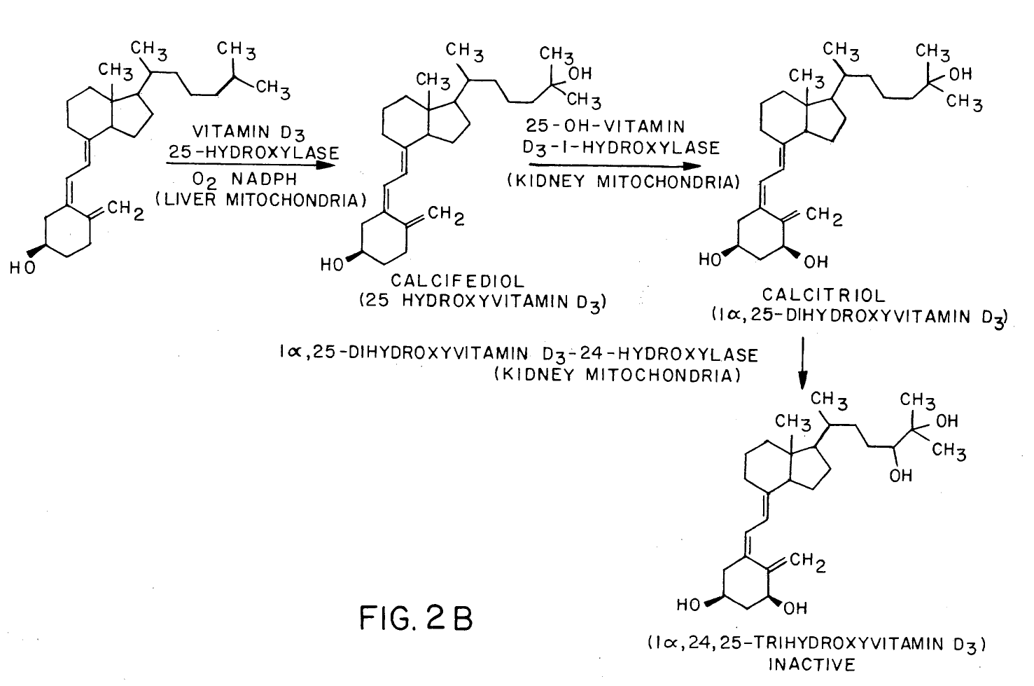

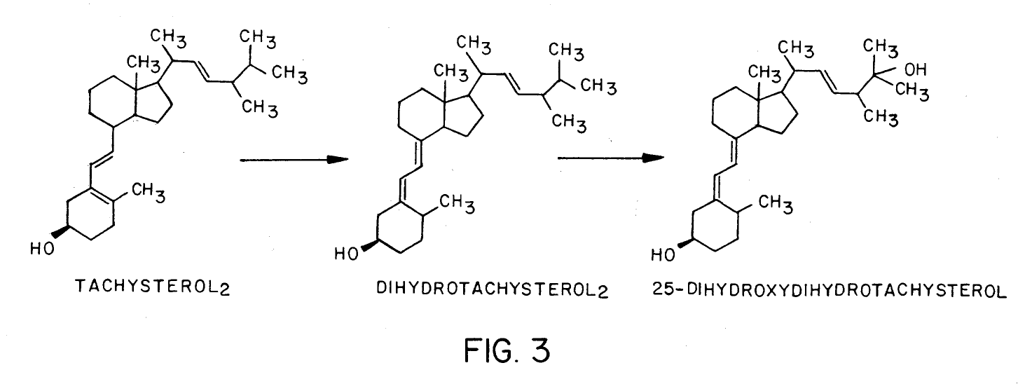

It is known that provitamin D, when irradiated by UVB generates essentially four products: cis-previtamin D, trans-previtamin D (tachysterol), lumisterol, and a minor proportion of a chemical group called toxisterols. (FIG. 2) The cis-previtamin D (usually called previtamin D) thermally isomerizes to vitamin D. In the skin, the remaining tachysterol, lumisterol, and toxisterols are generally incorporated into the plasma membranes of cells and sloughed off by the natural skin growth process.

Analysis of the reaction products of the UVB irradiation of provitamin D in a laboratory setting reveals the relative yields of the various compounds to be approximately 30% cis-previtamin D and 60-70% tachysterol (trans-previtamin D) with lumisterol and the toxisterols being only minor reaction products. In industrial chemical applications, the relative yields of the cis and trans forms is altered by the subsequent addition of a photosensitizer to the tachysterol and treatment with UVA irradiation. The photosensitizer plus UVA convert tachysterol to cis-previtamin D, which is thermally isomerized to vitamin D.

It is suggested that a similar reaction occurs in PUVA photochemotherapy. The photosensitizer psoralen plus UVA convert the tachysterol in the skin to cis-previtamin D that in turn thermally isomerizes to vitamin D (with body heat). However, the amount of calcitriol that can be produced in PUVA is limited by the amount of trans-previtamin D present in the cells. No therapies have been developed that include treatment with trans-previtamin D. PUVA cannot be used in conjunction with the application of vitamin D or calcitriol to the skin, as they would be destroyed during irradiation. Pretreatment with provitamin D before PUVA alone would be equally ineffectual as the provitamin D would not be converted to either cis- or trans-previtamin D in the absence of UVB irradiation. Isomerization of trans-previtamin D can also be carried out by the use of blue light in combination with a photosensitizing agent, however, exposure doses to blue light are substantially larger than that required for treatment with UVA.

In cases where the tissue is easily accessible (epidermis, dermis) both UVB and UVA are good candidate light treatment. These diseases and disorders include psoriasis, vitiligo, rosacea, alopecia, pemphigus, and inflammatory acne. In those cases where erythema or tanning (hyperpigmentation) may present a complication, blue light can be used and penetrates about 2-5 mm into the tissue.

Although UVB therapy may increase the production of vitamin D and subsequently calcitriol, the yield of both compounds is limited by the amount of provitamin D present in the tissue treated. No therapies have been developed which combine the application of a topical ointment containing provitamin D or any other precursor substance with UVB irradiation. No therapies have been designed to incorporate sequential administration of UV irradiation or blue light for the treatment of autoimmune diseases or cancer.

It is proposed that the instant invention ameliorates autoimmune disease and cancer by increasing the levels of calcitriol in the cell while releasing retinoids from RXR making RXR available for interaction with calcitriol and VDR. In the method, provitamin D is applied to the tissue of interest where it is able to reach relatively high and homogeneous levels as it is neither degraded by 24-hydroxylase nor cleared by VDBP into the circulation. UVB irradiation serves to activate the rearrangement of pro-vitamin D to two major products, cis- and trans-vitamin D. Once vitamin D is produced in the tissue, it is converted to calcitriol by cytochromes CYP 25, CYP27A and CYP27B, a large group of oxidizers called P450 cytochromes present in all cells. Simultaneously, UVB irradiation releases retinoids from RXR within the cells. This allows for the formation of the calcitriol-RXR-VDR and translocation of the complex to the nucleus where it is able to modulate transcription of inflammatory mediators by binding to NFAT and VDRE sites in the DNA. Optionally, UVA or blue light, particularly with the addition of photosensitizing agents, results in the isomerization of trans-previtamin D to cis-vitamin D to and therefore higher levels of vitamin D and finally calcitriol. The calcitriol-VDR-RXR heterotrimer is formed and translocates to the nucleus to reduce transcription of inflammatory mediators. Inhibitors to this process like NFkappaB and TNF-alpha are addressed in the invention by the antagonistic activity of vitamin E succinate (VES), vitamin C and other NFkappaB and TNF-alpha inhibitors to these endogenous inhibitors.

### EXAMPLE 1

Treatment of psoriasis with UVB irradiation. A female patient with a ½ inch round psoriatic lesion on the leg was treated with provitamin D (35 mg/gram ointment) and irradiated with UVB (3 MED). After 1 day the appearance was slightly indurated and red. Vitamin E succinate was applied (20 mg VES/gram ointment) and within 12 hours the tissue was not indurated and the redness disappeared. The formally lesional tissue looked like normal healthy tissue.

### EXAMPLE 2

Treatment with UVB irradiation. For topical disorders such as psoriasis, vitiligo, or atopic dermatitis, the disorder is largely confined to the epidermal region and the penetration of UVB wavelengths is sufficient to treat the area. A topical ointment containing 1 to 100 milligrams of 7-dehydrocholesterol per gram ointment and from 1 to 10 milligrams per gram ointment of VES is applied to the lesional area 12-18 hours prior to the irradiation with UVB. Application in the evening is recommended to allow time for the provitamin D to penetrate into the epidermis without UVB radiation in the environment. The area is irradiated with narrow band UVB radiation containing wavelengths between 290-315 nm. Wavelengths below 290 nm are preferably avoided. The dosage of the UVB radiation is preferably from a focused or targeted irradiation source using 2-8 minimum erythemal doses (MED's). Alternatively, the method can be performed with light sources that are-more limiting in their dosing capability (2 MED's or less) such as booths or hand held UVB devices. The patient is monitored for amelioration of symptoms and the therapeutic regimen is repeated as required.

### EXAMPLE 3

Treatment with UVB and other wavelengths. Treatment with two wavelengths of light begins as detailed in Example 2 with application of the topical application of provitamin D or a provitamin D analog such as 25 hydroxy 7-dehydrocholesterol to the area to be treated followed by irradiation with UVB (0.5-5 MED), and UVA (1-5 J/cm2) or blue light (2-20 J/cm2). The UVA or blue light is applied either concurrently with or following the UVB dose. The dose of UVA, with a wavelength of around 325-350 nm, is preferably administered from a focused irradiation source. Alternatively, UVA may be delivered using a booth or limb box. Blue light with strong radiation from 400-440 nm can be used in lieu of UVA. This dual irradiation strategy is particularly useful when the tissue characteristic become dose limiting to the UVB radiation (e.g. vitiligo).

A therapeutic regimen for UVB/UVA or UVB/blue light includes a lipophyllic photosensitizer such as anthracene or coal tar derivatives in the ointment base comprising 0.5% to 5% by weight of the ointment base. An example of such a topical ointment the formulation contains provitamin D (1-100 mg), anthracene (5-50 mg), and trans-previtamin D (10-250 mg) and VES (1-10 mg) per gram of ointment. The addition of tachysterol and photosensitizer is appropriate when the area treated is lacking in either previtamin or provitamin D like the hands or feet. After treatment with the method, the patient is monitored for amelioration of symptoms and the therapeutic regimen is repeated as required.

### EXAMPLE 4

Treatment with UVA irradiation. For very thick psoriasis plaques or phototherapy for psoriasis on the hands and soles of the feet, UVA radiation gives deeper dermal penetration. A topical ointment containing tachysterol (10-250 mg), VES (1-10 mg) and anthracene or coal tar (5-50 mg) per gram of ointment is applied to the skin several hours before treatment, preferably the night before treatment with UVA. The ointment covers about 1500 to 2000 sq. cm. of skin. Radiation preferably in the range from 320-400 nm is ideally delivered from a focused or targeted irradiation source using 2-4 minimum phototoxic doses (MPD's). Administration of blue light (400-440 nm) concurrent with or subsequent to the UVA radiation can be used without increasing the erythema of the UVA MPD dose. Alternatively a topical formulation containing only tachysterol and VES can be used in conjunction with oral or topical psoralen is applied per normal protocols prior to irradiation. The patient is monitored for amelioration of psoriasis and the treatment is repeated as required.

### EXAMPLE 5

Treatment with blue light. The formulation of the topical ointment for use with the treatment with blue light includes vitamin D (1-100 milligrams) or calcitriol (1-5 microgram) or calcitriol analog (1-50 microgram) and vitamin E succinate (1-10 mg) per gram ointment, which covers about 1500 to 2000 sq. cm. of skin. The ointment is applied to the lesional area 12-18 hours prior to the irradiation procedure, or can be infused into a solid tumor. Blue light irradiation (400-440 nm) is preferably delivered using a focused delivery device at a dose of 30-65 J/cm2. Alternatively a broad area illumination device like a gallium iodide lamp can be used as a source. The patient is monitored for the persistence of tumor.

Although an exemplary embodiment of the invention has been described above by way of example only, it will be understood by those skilled in the field that modifications may be made to the disclosed embodiment without departing from the scope of the invention, which is defined by the appended claims.

I claim:

1. A method for treatment of autoimmune disease and cancer comprising:
application of a composition comprising provitamin D to a site to be treated, followed by irradiation with UVB.

2. The method as in claim 1, wherein the provitamin D is selected from a group consisting of 7-dehydrocholesterol, ergosterol, and 1-alpha and /or 25-hydroxy substitutions thereto.

3. The method as in claim 1, wherein the provitamin D is an analog of provitamin D.

4. The method as in claim 1, wherein the provitamin D analog is selected from a group consisting of calcitriol precursors and calcitriol analog precursors.

5. The method as in claim 1, wherein the provitamin D analog is a calcipotriene analog.

6. The method as in claim 1, wherein the composition further comprises at least one member of a group consisting of tumor necrosis factor (TNF)-alpha inhibitors and fuclear factor (NF)kappaB inhibitors.

7. The method as in claim 1, wherein the composition further comprises at least on member of a group consisting of vitamin E succinate, n-acetylcysteine and prostaglandin E2.

8. The method as in claim 1, wherein the method further comprises oral administration of at least one member of a group consisting of TNF-alpha inhibitors and NFkappaB inhibitors.

9. The method as in claim 1, wherein the method further comprises oral administration of at least one member of a group consisting of etanercept and infliximab.

10. The method as in claim 1, wherein UVB irradiation comprises exposure to light having a wavelength of about 290-320 nm.

11. The method as in claim 1, wherein UVB irradiation comprises exposure to light having a wavelength of about 290-315 nm.

12. The method as in claim 1, wherein the method comprises irradiation with UVA after irradiation with UVB.

13. The method as in claim 12, wherein UVA irradiation comprises exposure to light having a wavelength of about 320-390 nm.

14. The method as in claim 12, wherein UVA irradiation comprises exposure to light having a wavelength of about 325-350 nm.

15. The method as in claim 12, wherein the method comprises the application of a photosensitizing agent before irradiation with UVA.

16. The method of claim 12, wherein the photosensitizing agent is selected from a group consisting of psoralen, psoralen analogs, anthracene, coal tar, coal tar derivatives, furocoumarins, and thiophene based pharmaceuticals.

17. The method as in claim 1, wherein the method comprises exposure to blue light after irradiation with UVB.

18. The method as in claim 17, wherein exposure to blue light comprises exposure to light having a wavelength of about 400-440 nm.

19. The method as in claim 18, wherein the method comprises the application of a photosensitizing agent before exposure to blue light.

20. The method of claim 19, wherein the photosensitizing agent is selected from a group consisting of psoralen, psoralen analogs, anthracene, coal tar or coal tar derivatives, furocoumarins, or thiophene based pharmaceuticals.

21. A method for the treatment of autoimmune disease and cancer comprising:
application of a composition comprising trans-previtamin D to a site to be treated,
followed by irradiation with UVA.

22. The method as in claim 21, wherein trans-previtamin D is selected from a group consisting of trans-previtamin D2, trans-previtamin D3, and previtamin D analogs thereof.

23. The method as in claim 21, wherein UVA irradiation comprises exposure to light having a wavelength of about 320-390 nm.

24. The method as in claim 21, wherein UVA irradiation comprises exposure to light having a wavelength of about 325-350 nm.

25. The method of claim 21, further comprising the application of a photosensitizing agent before irradiation with UVA.

26. The method of claim 25, wherein the photosensitizing agent is selected from a group consisting of psoralen, psoralen analogs, anthracene, coal tar, coal tar derivatives, furocoumarins and thiophene pharmaceuticals.

27. A method for the treatment of autoimmune disease and cancer comprising:
application of a composition comprising vitamin D to a site to be treated, followed by irradiation with blue light.

28. The method as in claim 27, wherein vitamin D is selected from a group consisting of cholecalciferol, 1-alpha cholecalciferol, 25 hydroxy cholecalciferol, 1,25 dihydroxycholecalciferol, ergocalciferol, 1-alpha ergocalciferol, 25 hydroxy ergocalciferol and 1,25 dihydroxyergocalciferol.

29. The method as in claim 27, wherein exposure to blue light comprises exposure to light having a wavelength of about 400-470 nm.

30. The method as in claim 27, wherein exposure to blue light comprises exposure to light having a wavelength of 400-440 nm.

31. A composition comprising a vitamin D precursor wherein the vitamin D precursor is selected from the group consisting of provitamin D, provitamin D analogs, trans-previtamin D and trans-previtamin D analogs in a formulation of a consistency that may be applied topically.

32. The composition of claim 31 further comprising at least one member of the group comprising vitamin E succinate, vitamin C, N-acetylcysteine.

33. The composition of claim 31 further comprising prostaglandin E2.
